(19) United States
(12) Patent Application Publication
(10) Pub. No.: US 2007/0299017 A1
(43) Pub. Date: Dec. 27, 2007

(54) COMPOSITIONS FOR LOWERING BLOOD SERUM CHOLESTEROL AND USE IN FOODS, BEVERAGES, AND HEALTH SUPPLEMENTS

(76) Inventors: Mitchell M. Kanter, Excelsior, MN (US); Melanie Jean Goulson, Dayton, MN (US); Bruce M. Resnick, Eden Prairie, MN (US)

Correspondence Address: CARGILL, INCORPORATED, LAW/24, 15407 MCGINTY ROAD WEST, WAYZATA, MN, 55391, US

(21) Appl. No.: 11/767,954

(22) Filed: Jun. 25, 2007

Related U.S. Application Data
(60) Provisional application No. 60/815,951, filed on Jun. 23, 2006.

(51) Int. Cl. A61K 31/70; A61K 31/352; A61P 9/00; A61K 31/56

(52) U.S. Cl. 514/23; 514/169; 514/182; 514/457; 514/685

(57) ABSTRACT

There is disclosed a composition comprising at least one of a compound selected from the group consisting of a bioflavonoid, a beta-1,3-beta-1,4-polymer of glucose having a weight average molecular weight (MW) of not greater than 1000 kDa, and a sterol. Moreover, there is disclosed a food or beverage or health supplement product comprising the disclosed composition. Further, there is disclosed a method of lowering blood serum cholesterol levels comprising administering to a patient in need thereof an effective amount of the disclosed composition.

### RELATED APPLICATION

This application claims the benefit of priority of U.S. provisional application No. 60/815,951, filed Jun. 23, 2006, the disclosure of which is hereby incorporated by reference.

### FIELD OF THE DISCLOSURE

The disclosure relates to compositions, such as those of natural components, which are useful in lowering blood serum cholesterol levels, and the use thereof in foods, beverages, and health supplements.

### BACKGROUND OF THE DISCLOSURE

It is well established that elevated levels of blood serum cholesterol is a major risk factor for coronary heart disease. Accordingly, it is useful to lower the blood serum cholesterol level to help to prevent coronary heart disease. Therefore, there is a continuing need to develop means for achieving reduction of blood serum cholesterol levels, such as new compositions of components. Preferably, the components are of natural origin, having a long history of use in food supply to obviate safety concerns.

### SUMMARY OF THE DISCLOSURE

In an aspect, there is disclosed a composition comprising at least one of a compound selected from the group consisting of a bioflavonoid, a a beta-1,3-beta-1,4-polymer of glucose having a weight average molecular weight (MW) of not greater than 1000 kDa, and a sterol.

Moreover, in another aspect, there is disclosed a food or beverage or health supplement product comprising a composition comprising at least one of a compound selected from the group consisting of a bioflavonoid, a a beta-1,3-beta-1,4-polymer of glucose having a weight average molecular weight (MW) of not greater than 1000 kDa, and a sterol.

Further, in an aspect, there is disclosed a method of lowering blood serum cholesterol levels comprising administering to a patient in need thereof an effective amount of a composition comprising at least one of a compound selected from the group consisting of a bioflavonoid, a a beta-1,3-beta-1,4-polymer of glucose having a weight average molecular weight (MW) of not greater than 1000 kDa, and a sterol.

### DESCRIPTION OF THE EMBODIMENTS

In an aspect, the description relates to a composition comprising at least one of a compound selected from the group consisting of a bioflavonoid, a beta-1,3-beta-1,4-polymer of glucose having a weight average molecular weight (MW) of not greater than 1000 kDa, and a sterol, that is suitable for use in lowering blood serum cholesterol levels. In an aspect, the composition can comprise a sterol and a beta-1,3-beta-1,4-polymer of glucose having a weight average molecular weight (MW) of not greater than 1000 kDa. In another aspect, the composition can comprise a sterol and at least one bioflavonoid. In a further aspect, the composition can comprise a bioflavonoid, and a beta-1,3-beta-1,4-polymer of glucose having a weight average molecular weight (MW) of not greater than 1000 kDa.

A food or beverage or health supplement product can comprise the disclosed composition. The food or beverage or health supplement product can be suitable for use in lowering blood serum cholesterol levels.

In another aspect, the disclosure relates to a method for lowering blood serum cholesterol levels comprising administering to a person in need thereof any one, or more, of the disclosed compositions and/or food or beverage or health supplement products described herein.

In another embodiment, the disclosure relates to a method for lowering blood serum cholesterol levels comprising administering to a patient in need thereof any one, or more, of the disclosed compositions and/or food and/or beverage or health supplement products described herein.

In more detail, the bioflavonoid utilized in the compositions, and in the food and beverage and health supplement products of the present disclosure is described as follows. Any bioflavonoid can be used. In another embodiment, the bioflavonoid used can be selected from the group consisting of chalcones; flavones such as apigenin, luteolin, polymethoxylated flavones and the like; flavonols such as quercitol, kaempferol, myricetin, and the like; flavanones; anthocyanins such as resveritrol and the like; isoflavanoids such as daidzein, genestiein and the like; and the like. Also suitable bioflavonoids include nobiletin, tangeretin (also known as tangeritin), mixtures thereof, and the like. Mixtures of any of the bioflavonoids can be used.

In another aspect, the bioflavonoid used can be a citrus flavonoid. These citrus flavanoids can be found in citrus foods, such as oranges, grapefruits, tangerines, and the like. Non-limiting examples of citrus flavanoids include nobiletin, tangeretin, sinensetin, hesperidin, maringin, maringenin, hesperetin, and the like. Mixtures of the bioflavonoids can be used. These materials are also known in the art as bioflavonoids, bioflavinoids, bioflavenoids, flavonoids, flavanoids, flavinoids, or flavenoids.

Suitable for use in an aspect, in the compositions, and food and beverage and health supplement products disclosed herein, are beta-1,3-beta-1,4-polymer of glucose having a weight average molecular weight (MW) of not greater than 1000 kDa. In another aspect, the polymer of glucose has a weight average molecular weight (MW) ranging from about 100 to about 250 kDa, and in yet another embodiment from about 120 to about 170 kDa. See United States Patent Application Publication No. 2004/0258829A1, published Dec. 23, 2004, the disclosure of which is hereby incorporated by reference in its entirety, for a description of the polymer and its method of preparation.

In paragraph [0122] of the published application, there is described the method for determining the weight average molecular weight (MW) of the beta-1,3-beta-1,4-polymer of glucose utilized herein. The procedure is reproduced as follows:

A 20 mg sample of finely milled beta-glucan (<0.25 mm) was added to a 50 mL glass test tube followed by addition of 100 microliters of 95% (v/v) ethanol. 20 mL of filtered (0.2 microns) ultra-pure water was added to the test tube with vortexing. The sample was heated for 1 hour in boiling water with occasional mixing. The sample was filtered (0.45 microns) into a liquid chromatograph vial and is then injected. Size Exclusion Chromatography (SEC) coupled with Multi-Angle Laser Light Scattering (MALLS, Dawn EOS, Wyatt Technologies Inc.) and Refractive Index (RI, Waters 410) detectors was used to determine the weight average molecular weight distribution of the beta-glucan. 100 microliters of sample was injected onto the SEC columns (Shodex OH-pak SB-G/805/804/803) via a Waters 2690 HPLC system. The columns were run at 40° C. with a flow rate of 1.0 mL/min and a mobile phase (pre filtered, 0.1 microns) of 200-ppm sodium azide in water. The MALLS detector uses Astra Software (Version 4.73.04) with a dn/dc value for beta glucan of 0.150. A Debye plot was used to calculate the weight average molecular weight distribution.

Any sterol can be used in the compositions herein that are suitable for lowering blood serum cholesterol levels. The term sterol includes, but is not limited to any sterol, any stanol, and mixtures thereof; also included, but not limited to, are esters of sterols, esters of stanols, and mixtures thereof. The esters can be carboxylic acid esters such as fatty acid esters. The sterol can be any sterol obtained from a vegetable, a soybean, a tree, and mixtures thereof.

Plant sterols are suitable for use herein as the sterol. The term “plant sterol” includes, without limitation, phytosterols, phytosterol esters, phytostanols, and phytostanol esters.

Plant sterols for use herein can include any of various positional isomer and stereoisomeric forms, such as α-, β-, or γ-isomers. Typical phytosterol compounds include α-sitosterol, γ-sitosterol, β-sitosterol, campesterol, stigmasterol, brassicasterol, spinosterol, taraxasterol, desmosterol, chalinosterol, poriferasterol, clionasterol, ergosterol, Δ-5 avenosterol, Δ-5-campesterol, clerosterol, Δ-5-stigmasterol, Δ-7, 25-stigmadienol, Δ-7-avenosterol, Δ-7-β-sitosterol, and Δ-7-brassicasterol.

Suitable examples of phytosterol esters include, without limitation, β-sitosterol laurate ester, α-sitosterol laurate ester, γ-sitosterol laurate ester, campesterol myristearate ester, stigmasterol oleate ester, campesterol stearate ester, β-sitosterol oleate ester, β-sitosterol palmitate ester, β-sitosterol linoleate ester, α-sitosterol oleate ester, γ-sitosterol oleate ester, β-sitosterol myristearate ester, β-sitosterol ricinoleate ester, campesterol laurate ester, campesterol ricinoleate ester, campesterol oleate ester, campesterol linoleate ester, stigmasterol linoleate ester, stigmasterol laurate ester, stigmasterol caproate ester, α-sitosterol stearate ester, γ-sitosterol stearate ester, α-sitosterol myristearate ester, γ-sitosterol palmitate ester, cam pesterol ricinoleate ester, stigmasterol ricinoleate ester, campesterol ricinoleate ester, and stigmasterol stearate ester.

Useful phytostanol compounds include α-, β-, and γ-sitostanol, campestanol, stigmastanol, spinostanol, taraxastanol, brassicastanol, desmostanol, chalinostanol, poriferastanol, clionastanol, and ergostanol.

Finally, phytostanol esters for inclusion in a composition provided herein include, without limitation, β-sitostanol laurate ester, campestanol myristearate ester, stigmastanol oleate ester, campestanol stearate ester, β-sitostanol oleate ester, β-sitostanol palmitate ester, β-sitostanol linoleate ester, β-sitostanol myristearate ester, β-sitostanol ricinoleate ester, campestanol laurate ester, campestanol ricinoleate ester, campestanol oleate ester, campestanol linoleate ester, stigmastanol linoleate ester, stigmastanol laurate ester, stigmastanol caproate ester, stigmastanol stearate ester, -α-sitostanol laurate ester, γ-sitosterol laurate ester, α-sitostanol oleate ester, γ-sitosterol oleate ester, α-sitostanol stearate ester, γ-sitosterol stearate ester, α-sitostanol myristearate ester, γ-sitosterol palmitate ester, campestanol ricinoleate ester, stigmastanol ricinoleate ester, campestanol ricinoleate ester, β-sitostanol, α-sitostanol, γ-sitosterol, campestanol, and stigmastanol.

Steryl esters are suitable for use herein, and include, but are not limited to, fatty acid esters of plant phytosterols such as: sitosterol, campesterol, stigmasterol, brassicasterol, avenasterols, and diosgenin, or mixtures thereof. The sterol esters also include the esterified and hydrogenated forms of sterols such as sitostanol and campestanol, and the like, ferulate esters, or succinate esters.

Typical of those suitable for use in the present compositions are α, β-, γ-sitosterol, stigmasterol, ergosterol, campesterol, avenasterol, brassicasterol, desmosterol, chalinosterol, poriferasterol, clionasterol, sitostanol, stigmastanol, campesterol or a mixture of one or more of the above phytosterols or phytostanols.

The compositions of the present disclosure are prepared by using any manner. For example, the ingredients of the compositions may be physically admixed together.

In one embodiment, the composition can comprise at least one compound selected from the group consisting of a bioflavonoid, a beta-1,3, beta-1,4-polymer of glucose having a weight average molecular weight (MW) of not greater than 1000 kDa, and a sterol, wherein the at least one compound is admixed in any amounts and any combinations to form a composition that is suitable for lowering blood serum cholestserol levels. In an aspect, the at least one compound can be present in any amount suitable for lowering blood serum cholesterol levels. For example, the bioflavonoid can be present in the composition in an amount ranging from about 0.05% to about 87%, for example from about 0.07% to about 80%, as a further example from about 10% to about 60%, and as another example from about 15% to about 50% by weight, relative to the total weight of the composition.

For example, the beta-1,3-beta-1,4-polymer of glucose having a weight average molecular weight (MW) of not greater than 1000 kDa can be present in the composition in an amount ranging from about 10% to about 97%, for example from about 10% to about 95%, as a further example from about 15% to about 95%, and another example from about 15% to about 92% by weight, relative to the total weight of the composition.

Moreover, the sterol can be present in the composition in an amount ranging from about 3% to about 90%, for example from about 5% to about 90%, as a further example from about 5% to about 85%, another example from about 8% to about 85% by weight, relative to the total weight of the composition. In an aspect, the sterol can be present in an amount of from about 40% to about 90%, for example from about 50% to about 85% by weight relative to the total weight of the composition.

All of the compositions described herein have blood serum cholesterol lowering properties, and can be incorporated into any food product, or beverage, or health supplement.

Exemplary food products into which the blood serum cholesterol lowering compositions may be incorporated include, but are not limited to bakery products such as bread, rolls, cake, muffins, waffles and the like, biscuits, cookies, crackers, and the like; cereal products such as breakfast cereals, enriched flours, pasta products, snacks and the like; bran products; beverages such as alcoholic and non-alcoholic drinks, juices, dietary supplements and the like; dairy products such as milk based products, yogurt, ice cream, desserts, cheese, and the like, or non-dairy products such as desserts and the like; ready mixes; meat products, egg products, spreads, salad dressing, oils, mayonnaise, and the like.

Other suitable examples of food and beverage applications into which the blood serum cholesterol lowering compositions of the present disclosure may be incorporated are as follows:
Supplement Powders, Chews, and Confections
Juice (condensed) and ready to drink (RTD)
Juice Drinks (RTD, Condensed, Instant)
Milk, (Dairy, Soy, Rice) and Milk-Based Beverages
Instant Protein Smoothies, Shakes and Meal Replacements
RTD Smoothies, Shakes and Meal Replacements
Protein Bars (cold extruded)
Baked Bars (including fruit filled)
Healthy Snacks, Candy and Confections
Granola/Cereal/Trail Mix/Snack Bars
RTE Cereal (extruded), ready to eat (RTE)
Hot Cereal
Soup (Instant, Condensed, RTE)
Yogurt (Dairy, Soy), Puddings
Sauces
Soft Drinks, Instant RTD (carbonated and non carbonated)
Coffee, Coffee Based Beverages, and Creamers (instant and liquid)
Alcoholic beverages

The food and beverage and health supplement products comprise sufficient amounts of the blood serum cholesterol lowering compositions to provide a reduction in blood serum cholesterol levels.

It has also been found that use of the combinations of compounds produce an improved lowering of blood serum cholesterol levels than results from use of the individual ingredients, under the parameters of the study herein.

The present disclosure also relates to a method of lowering blood serum cholesterol levels by administering an effective amount of any of the blood serum cholesterol lowering compositions described herein, or any of the foods and beverages and health supplements that comprise the blood serum cholesterol lowering compositions. In one aspect, the blood serum cholesterol lowering compositions, or foods and/or beverages and/or health supplements comprising the blood serum cholesterol lowering compositions, are administered orally.

The following examples are presented to illustrate the present disclosure and to assist one of ordinary skill in making and using the same. The examples are not intended in any way to otherwise limit the scope of the disclosure.

TEST PROCEDURES—The following test procedure was utilized in evaluating the effectiveness of the compositions of the present disclosure in lowering blood serum cholesterol.

In the evaluation, male broiler chickens of the Ross 308 strain were used as the test subjects. The broilers were fed diets that were formulated to be adequate in all nutrients. The positive control diet contained 1% cholesterol. The ingredients being tested were a soy phytosterol ester; a barley beta glucan that was a beta-1,3-beta 1,4-polymer of glucose having a weight average molecular weight (MW) ranging from about 120 to about 170 kDa; and a citrus flavonoid mixture comprising 13.80% nobiletin and 15.10% tangeretin.

The study period included an initial 7 days standardization period, followed by a 7 day loading period during which diets containing 1% cholesterol were fed ad libitum diet to all groups except the negative control. On day 15 of the trial, the test ingredients, namely, the soy phytosterol ester, the barley beta glucan, and the citrus flavanoid mixture, were introduced into the diet as individual ingredients, and as combinations of 2 and 3 ingredients.

The diet included yellow no. 2 corn, soybean meal, corn oil, salt, calcium carbonate, mono-dicalcium phosphate, choline chloride, D, L, —methionine, poultry vitamin, poultry trace mineral, and sand. The optional ingredients were cholesterol, soy phytosterol ester, barley beta glucan and the 29% flavonoid mixture of nobiletin (13.80%) and tangeretin (15.10%).

In the study, the broilers were fed compositions containing 0.5% soy phytosterol ester, 0.75% barley beta glucan having a weight average molecular weight (MW) of not greater than 1000 kDa, and 0.075% citrus flavonaid mixture, as individual ingredients, or as combinations of two (2) ingredients, or as a combination of all three ingredients. There were eight (8) treatment groups.

On day 42 of the study, the feed was withdrawn from the broilers, and the broilers were immediately weighed. After a minimum of 6 hours following withdrawal of feed, blood was collected from the brachial vein from 3 broilers per pen, and placed in 12×75 mm polypropylene tubes. Blood was placed on ice immediately after collection to prevent compositional changes prior to delivery to the laboratory for processing. Blood was centrifuged for separation of serum, and placed in microfuge tubes for shipment to Marshfield Laboratories for determination of cholesterol concentration. The cholesterol samples were analyzed using a Roche Diagnostic Modular Analyzer, that utilizes both P and D modules. Cholesterol was assayed utilizing a Roche diagnostic cholesterol—HP assay kit (catalog no. 1875523). The cholesterol values are expressed as mg cholesterol/dL of blood serum in Table II.

In addition to serum cholesterol concentrations, other evaluation criteria included weight gain, feed intake, and feed efficiency. All data were analyzed by the GLM procedure (SAS 2001) as a complete randomized block design. LSMeans analysis was conducted to separate treatment effects.

During the experimental period, the broiler chickens were fed eight (8) different diet formulations for 35 days. The detailed compositions of the diet formulations are shown in Table I. All amounts of the ingredients are expressed as % by weight in Table I.

TABLE I
Diet Formulations

| | Diet | | | | | | | |
|---|---|---|---|---|---|---|---|---|
| Ingredients | 1 | 2 | 3 | 4 | 5 | 6 | 7 | 8 |
| Yellow #2 corn | 61.418 | 61.418 | 61.418 | 61.418 | 61.418 | 61.418 | 61.418 | 61.418 |
| Soybean Meal | 29.932 | 29.932 | 29.932 | 29.932 | 29.932 | 29.932 | 29.932 | 29.932 |
| Corn Oil | 2.615 | 2.615 | 2.615 | 2.615 | 2.615 | 2.615 | 2.615 | 2.615 |
| Salt | 0.436 | 0.436 | 0.436 | 0.436 | 0.436 | 0.436 | 0.436 | 0.436 |
| Calcium Carbonate | 1.367 | 1.367 | 1.367 | 1.367 | 1.367 | 1.367 | 1.367 | 1.367 |
| Mono-dicalcium | 1.395 | 1.395 | 1.395 | 1.395 | 1.395 | 1.395 | 1.395 | 1.395 |
| phosphate | | | | | | | | |
| Choline Chloride | 0.018 | 0.018 | 0.018 | 0.018 | 0.018 | 0.018 | 0.018 | 0.018 |
| D,L-methionine | 0.140 | 0.140 | 0.140 | 0.140 | 0.140 | 0.140 | 0.140 | 0.140 |
| Poultry vitamin | 0.125 | 0.125 | 0.125 | 0.125 | 0.125 | 0.125 | 0.125 | 0.125 |
| Poultry trace mineral | 0.050 | 0.050 | 0.050 | 0.050 | 0.050 | 0.050 | 0.050 | 0.050 |
| Sand | 2.500 | 1.500 | 1.000 | 0.750 | 1.275 | 0.250 | 0.775 | 0.025 |
| Cholesterol | — | 1.500 | 1.000 | 1.000 | 1.000 | 1.000 | 1.000 | 1.000 |
| Phytosterol Ester 91% | — | — | 0.500 | — | — | 0.500 | 0.500 | 0.500 |
| β-glucan, 70% | — | — | — | 0.750 | — | 0.750 | — | 0.750 |
| Flavonoid, 33% | — | — | — | — | 0.075 | — | 0.075 | 0.075 |
| Total | 100.00 | 100.00 | 100.00 | 100.00 | 100.00 | 100.00 | 100.00 | 100.00 |

The cholesterol determinations were carried out as described herein on the blood samples taken from the broilers fed with each of the 8 diet formulations of Table I. The cholesterol determinations are reported in Table II, utilizing experiment numbers corresponding to the experiment numbers of Table I. The cholesterol data is expressed as mg cholesterol/dL blood serum. As mentioned earlier, the determinations were made after a 42 day study period.

TABLE II
Serum Cholesterol Concentrations

| | | Cholesterol | % change |
|---|---|---|---|
| | | Level, | vs. positive |
| | Dietary Treatment | mg/dL | control |
| 1 | Negative control (corn-soy-corn oil) | 131 | — |
| 2 | Positive control (cholesterol 1%) | 262 | — |
| 3 | Phytosterol ester + cholesterol | 258 | −2 |
| 4 | β-glucan + cholesterol | 302 | 15 |
| 5 | Citrus flavonoids + cholesterol | 274 | 5 |
| 6 | Phytosterol ester + β-glucan + | 217 | −17 |
| | cholesterol | | |
| 7 | Phytosterol ester + Citrus flavonoids + | 185 | −29 |
| | cholesterol | | |
| 8 | Phytosterol ester + β-glucan + Citrus | 210 | −20 |
| | flavonoids + cholesterol | | |

From the above data, it is observed that use of any of the ingredients individually, in the quantity fed, resulted in substantially no significant change in cholesterol level, as compared to the positive control. Consequently, none of the individual ingredients was effective in the quantities consumed in this study, in achieving the objective of lowering blood serum cholesterol.

It has now been found, unexpectedly, that certain combinations of the individual ingredients in the amounts fed, that did not lower blood serum cholesterol levels, when combined in specific combinations exhibit a lowering of the blood serum cholesterol levels, in amounts ranging from 17 to 29%, as compared to the positive control. It is apparent, then, from the data in the Tables I and II, that the combinations of the ingredients described herein, provide enhanced blood serum cholesterol lowering effect as compared to use of the individual ingredients separately, under the parameters of the study.

Although the data in the Tables I and II are based on experiments using broiler chickens, it is expected that the compositions, and foods or beverages or health supplements comprising the compositions, would be similarly effective in lowering blood serum cholesterol levels in mammals such as humans.

The blood serum cholesterol lowering compositions described herein can be suitable for use in providing foods and beverages and health supplements that result in a lowering of blood serum cholesterol levels. Any food or beverage and/or health supplement can be combined with the blood serum cholesterol lowering compositions. Many examples of suitable foods and beverages and/or health supplements have been described herein.

The following are specific examples of foods and beverages containing certain combinations of ingredients, that when incorporated into the food or beverage, are expected to lower blood serum cholesterol levels.

### EXAMPLE 1

### Heart Healthy Juice Drink

In this example, there is described a heart healthy juice drink. The combination of ingredients herein comprises 0.04 g CoroWise™ DV-ES-100 plant sterols, available from Cargill, Inc., Minnesota; 0.75 g barley beta-1,3-beta-1,4 polymer of glucose having a weight average molecular weight (MW) ranging from about 120 to about 170 kDa available from Cargill; and 10.0 mg of citrus flavanoids from Sytrino available from Source Nutrition per 8 fluid ounce (240 g) serving of juice drink.

The formula for the heart healthy drink, in detail, and the process for producing the juice drink are as follows:

| Ingredient | % |
|---|---|
| Water | 96.635 |
| Orange Juice Concentrate (65 Brix, Cargill) | 1.4 |
| Pineapple Juice Concentrate (65 Brix, Cargill) | 0.70 |
| Barley Beta-Glucan (Cargill), 70% Beta Glucan | 0.45 |
| Plant Sterols (Coro Wise ™ DV-ES-100, Cargill) 50% Sterols | 0.34 |
| Sucralose Powder (McNeil Nutritionals) | 0.01 |
| Acesulfame K Powder (Nutrinova) | 0.012 |
| Citric Acid (Cargill) | 0.18 |
| Flavors | 0.15 |
| Potassium Citrate (Cargill) | 0.10 |
| Beta-Carotene (1% CWS, BASF) | 0.01 |
| Citrus Flavonoids (Sytrinol, Source Nutrition), 33% Flavonoids | 0.013 |
| Total | 100.00 |

Procedure: The water can be heated to about 90° C. Slowly sprinkle barley beta-glucan into the vortex of the water using high shear mixing, mixing for 15 mins. Add the plant sterols and continue to mix for 5 minutes. Add fruit juice concentrate, sweeteners, acidulants, flavor, citrus flavonoids, and color, and mix for 5 minutes. Adjust the pH to 3.2 with citric acid. Thermally process beverage and fill bottles.

### EXAMPLE 2

### Heart Healthy Instant Protein Drink

In this example, there is provided a heart healthy instant protein drink. The combination of ingredients utilized herein to lower blood serum cholesterol level comprises 0.65 g CoroWise™ Instant plant sterol esters, available from Cargill, Inc., of Minnesota; and 0.75 g of barley beta-1,3-beta-1,4-polymer of glucose having a weight average molecular weight (MW) ranging from about 120 to about 170 kDa, per 41.6 g serving of beverage powder, which may be stirred into water to make a beverage.

The formula for the heart healthy instant protein drink, in detail, and the process for producing the instant protein drink are as follows:

| Ingredient | % |
|---|---|
| Fructose, Crystalline (Tate & Lyle) | 47.49 |
| Soy Protein Isolate (Prolisse ™ 801, Cargill); 87% Soy Protein | 36.88 |
| Plant Sterol Esters (Coro Wise ™ Instant, Cargill) 33% Sterol | 4.81 |
| Ester | |
| Vitamin/Mineral Premix | 3.53 |
| Barley Beta-Glucan (Cargill), 70% Beta Glucan | 2.64 |
| Sugar | 2.25 |
| High Oleic Sunflower Oil (Cargill) | 2.05 |
| Flavors | 0.27 |
| Carboxymethylcellulose (Hercules) | 0.08 |
| Total | 100.00 |

Procedure: Dry blend all ingredients together, mixing until uniformly dispersed. Package as desired in individual serving packets or multi-serving canisters.

Beverage Preparation Instructions: Add 41.6 g powder to 8 fl.oz. cold water. Shake or blend 20 seconds or until well mixed.

### EXAMPLE 3

### Heart Healthy Dairy Yogurt

In this example, there is provided a heart healthy dairy yogurt. The combination of ingredients utilized herein to lower blood serum cholesterol levels comprise 0.65 g plant sterol esters from Coro Wise TMSE-C100 available from Cargill, Inc., Minnesota; and 81 mg of citrus flavonoids from Sytrinol available from Source Nutrition, per 225 g serving of yogurt.

The formula for the heart healthy dairy yogurt, in detail, and the process for producing the dairy yogurt are as follows:

| | % |
|---|---|
| White Mass Ingredients | |
| Skim Milk | 76.996 |
| Nonfat Dry Milk - Low Heat (Dairy America) | 4.00 |
| Sugar | 3.00 |
| Plant Sterol Esters (Coro Wise ™ SE-C100, Cargill) 91.0% | 0.32 |
| Sterol Ester | |
| Gelatin, Bloom 225 (PB Leiner) | 0.40 |
| Pectin, Low Methoxy (Cargill) | 0.17 |
| Yogurt Culture (DPLABY-2C Quick Start, Danisco) | 0.004 |
| Citrus Flavonoids (Sytrinol, Source Nutrition), 33% Flavonoids | 0.11 |
| White Mass Total | 85.0 |
| Fruit Preparation Ingredients | |
| Strawberry Puree (Fruitcrown) | 15.00 |
| Fruit Preparation Total | 15.00 |

Procedure: Premix the dry ingredients, except culture. Warm sterol esters until fluid and blend into warm milk. Add dry ingredients and blend. Homogenize milk blend: 2 stage, 2000/500 psi. Pasteurize at 185-190° F. for 30 minutes. Chill to 100-108° F. Add culture per supplier's recommendation. Incubate at recommended temperature (about 106-110° F.) until batch reaches pH 4.45 to 4.55. Break set by gently blending for 30 seconds. Quickly chill to 70-0° F. Gently blend the chilled white mass with the fruit preparation. Package and refrigerate.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A composition comprising:
at least one of a compound selected from the group consisting of a bioflavonoid, a a beta-1,3-beta-1,4-polymer of glucose having a weight average molecular weight (MW) of not greater than 1000 kDa, and a sterol.

2. The composition of claim 1, wherein the at least one bioflavonoid is selected from the group consisting of a chalcone, a flavone, a flavonol, a flavanone, an anthocyanin, an isoflavanoid, and mixtures thereof.

3. The composition of claim 1, wherein the bioflavonoid is a polymethoxylated flavone.

4. The composition of claim 1, the bioflavonoid is selected from the group consisting of nobiletin, tangeretin, and mixtures thereof.

5. The composition of claim 1, wherein at least one of the compound are present in amounts sufficient to lower blood serum cholesterol levels.

6. The composition of claim 1, wherein the bioflavonoid is present in an amount ranging from about 0.05% to about 87% by weight relative to the total weight of the composition.

7. The composition of claim 1, wherein the bioflavonoid is present in an amount ranging from about 0.07% to about 80% by weight relative to the total weight of the composition.

8. The composition of claim 1, wherein 1 wherein the beta-1,3-beta-1,4-polymer of glucose has a weight average molecular weight (MW) ranging from about 100 to about 250 kDa.

9. The composition of claim 1, wherein 1 wherein the beta-1,3-beta-1,4-polymer of glucose has a weight average molecular weight (MW) ranging from about 120 to about 170 kDa.

10. The composition of claim 1, wherein the beta-1,3-beta-1,4-polymer of glucose is present in an amount ranging from about 10% to about 97% by weight relative to the total weight of the composition.

11. The composition of claim 1, wherein the beta-1,3-beta-1,4-polymer of glucose is present in an amount ranging from about 10% to about 95% by weight relative to the total weight of the composition.

12. The composition of claim 1, wherein the sterol is a sterol obtained from a vegetable, a soybean, a tree, or mixtures thereof.

13. The composition of claim 1, wherein the sterol is selected from the group consisting of phytosterols, phytosterol esters, phytostanols, phytostanol esters, and mixtures thereof.

14. The composition of claim 1, wherein the sterol is present in an amount ranging from about 3% to about 90% by weight relative to the total weight of the composition.

15. The composition of claim 1, wherein the sterol is present in an amount ranging from about 5% to about 90% by weight relative to the total weight of the composition.

16. The composition of claim 1, wherein the composition comprises the bioflavonoid and the beta-1,3-beta-1,4-polymer of glucose.

17. The composition of claim 1, wherein the composition comprises the bioflavonoid and the sterol.

18. The composition of claim 1, wherein the composition comprises the beta-1,3-beta-1,4-polymer of glucose and the sterol.

19. A food or beverage or health supplement product comprising:
a composition comprising:
at least one of a compound selected from the group consisting of a bioflavonoid, a a beta-1,3-beta-1,4-polymer of glucose having a weight average molecular weight (MW) of not greater than 1000 kDa, and a sterol.

20. A method of lowering blood serum cholesterol levels comprising administering to a patient in need thereof an effective amount of a composition comprising:
at least one of a compound selected from the group consisting of a bioflavonoid, a a beta-1,3-beta-1,4-polymer of glucose having a weight average molecular weight (MW) of not greater than 1000 kDa, and a sterol.
